(19) United States
(12) Patent Application Publication
(10) Pub. No.: US 2025/0191775 A1
(43) Pub. Date: Jun. 12, 2025

(54) NEURO-OPHTHALMIC RISK ASSESSMENT

(71) Applicant: Locaze LLC, Arlington, MA (US)

(72) Inventors: Sagar Rastogi, Arlington, MA (US); Kedar Krishnan, Horsham, PA (US); Emma Anderson, Palatine, IL (US); Siddhi Date, Lansdale, PA (US); Ataes Aggarwal, Morgantown, WV (US)

(21) Appl. No.: 18/972,781

(22) Filed: Dec. 6, 2024

Related U.S. Application Data
(60) Provisional application No. 63/608,258, filed on Dec. 9, 2023.

(51) Int. Cl. G16H 50/30; A61B 5/00; A61B 5/16; G06T 7/00; G16H 10/60

(52) U.S. Cl. CPC G16H 50/30; A61B 5/0077; A61B 5/163; A61B 5/7267; A61B 5/7275; G06T 7/0012; G16H 10/60; G06T 2207/20081; G06T 2207/30041; G06T 2207/30201

(57) ABSTRACT

The present disclosure relates to a system, a method, and a computer program product for neuro-ophthalmic risk assessment(s) of a user. The method includes receiving facial image(s) of the user from a user device. The method further includes determining parametric value(s) associated with at least one facial feature of the user, based on the facial image(s) using neuro-ophthalmic test(s). Furthermore, the method includes determining a measure of risk for the user based on the parametric value(s) using Machine Learning (ML) models. The measure of risk indicates a level of neuro-ophthalmic risk associated with the user.

### CROSS REFERENCE TO RELATED APPLICATION

This application claims under 35 USC 120 the benefit of the filing date of U.S. Provisional Application 63/608,258, filed on Dec. 9, 2023, the entire content of which is incorporated herein by reference.

### TECHNICAL FIELD

The embodiments of the present disclosure generally relate to the field of healthcare and medical assessment. More particularly, the present disclosure relates to neuro-ophthalmic risk assessment.

### BACKGROUND

The subject matter disclosed in the background section should not be assumed or construed to be prior art merely due to its mention in the background section. Similarly, any problem statement mentioned in the background section or its association with the subject matter of the background section should not be assumed or construed to have been previously recognized in the prior art.

Neuro-ophthalmic impairments occur due to irregular functioning of the nervous system. Majority of the neuro-ophthalmic impairments occur due to an injury impacting brain functioning. Specifically, concussions are resultants of Traumatic Brain Injuries (TBIs) which often involve temporary neuro-ophthalmic impairment resulting from head impacts. The prevalence of concussions has increased even after the recent surge in awareness of TBIs. This increase in number of concussions partly reflect the ongoing difficulties in accurate and timely concussion identification. Presently, more than 50% of the concussions go undiagnosed which results in patients deprived of essential medical attention until they develop noticeable physical, mental, emotional, or behavioral symptoms. Delayed diagnosis and intervention not only worsen these symptoms but also increase the risk of prolonged brain damage.

Contemporary products available for concussion assessment rely on headgears. Multiple sensors are attached to the headgear which measure force of impact and do not provide any other relevant information to assess the injury. Since some factors, such as location or angle of impact, are not considered while detection of concussions, such products lack reliability in accuracy of concussion detection.

Some other solutions rely on a test or two to determine the severity of the concussion, which may be misleading as every person has a different initial response to a concussion. Majority of the solutions rely only on saccadic eye movements-based tests, which only affects 30% of the concussion patients. These tests are longer to execute and do not provide an accurate diagnosis. Moreover, some concussion assessment products and approaches rely on an external hardware, which reduces the accessibility and makes the procedure more complex. Majority of approaches focus on detection of generic impairments such as influence of alcohol or drugs, detection of cognitive impairment, etc. and are not specifically designed precisely for neuro-ophthalmic assessment. The lack of specificity of diagnosing concussions makes them inaccurate. Therefore, there is a need for a technical solution to overcome the abovementioned challenges.

### SUMMARY

The following embodiments present a simplified summary in order to provide a basic understanding of some aspects. This summary is not an extensive overview, and it is not intended to identify key/critical elements or to delineate the scope thereof. Its sole purpose is to present some concepts in a simplified form as a prelude to the more detailed description that is presented later.

According to an embodiment, a method for neuro-ophthalmic risk assessment(s) of a user is provided. The method includes receiving at least one facial image of the user from a user device. The method further includes determining parametric value(s) associated with at least one facial feature of the user, based on the at least one facial image through one or more neuro-ophthalmic tests. Furthermore, the method includes determining a measure of risk for the user based on the one or more parametric values using one or more Machine Learning (ML) models. The measure of risk indicates a level of neuro-ophthalmic risk associated with the user.

In some aspects of the present disclosure, the method further includes identifying one or more impairment levels for one or more impairments based on the one or more parametric values using the one or more ML models. The one or more impairments are associated with the one or more neuro-ophthalmic tests.

In some aspects of the present disclosure, the at least one facial feature of the user is associated with tracking a movement of eyes of the user and estimating a gaze of the eyes of the user.

In some aspects of the present disclosure, the method further includes determining, using the one or more parametric values through the one or more ML models, one or more risk scores corresponding to the one or more neuro-ophthalmic tests. Moreover, the method includes determining whether at least one risk score from the one or more risk scores mismatches with a non-impaired range for a corresponding neuro-ophthalmic test from the one or more neuro-ophthalmic tests. Furthermore, the method includes identifying at least one oculomotor impairment from the one or more impairments based on the determination that the at least one risk score mismatches with the non-impaired range.

In some aspects of the present disclosure, the method further includes generating a non-impaired report based on the determination that each risk score (from the one or more risk scores) matches with the corresponding non-impaired range.

In some aspects of the present disclosure, the one or more neuro-ophthalmic tests comprises at least one of an accommodation test to measure an eye focus adjustment of the user, a Vestibulo-ocular Reflex (VOR) test to measure a stability of the eyes of the user, a saccadic eye movement test to evaluate a rapid gaze shift of the user, a smooth pursuit test to analyze continuous object tracking by the user, and an optokinetic nystagmus test to analyze response to a succession of moving stimuli on the user.

In some aspects of the present disclosure, the one or more ML models are trained for determining a user feature metrics corresponding to one or more neuro-ophthalmic tests based on the parametric value(s) and comparing the user feature metrics with a baseline feature metrics to determine the one or more risk scores.

In some aspects of the present disclosure, the method further includes determining, based on the comparison of the user feature metrics with the baseline feature metrics, one or more data elements associated with information of the at least one oculomotor impairment. Moreover, the method includes generating a risk assessment report based on the one or more data elements. Furthermore, the method includes storing the risk assessment report with a risk assessment timestamp in a database. Furthermore, the method includes transmitting the risk assessment report to the user device.

In some aspects of the present disclosure, the method further includes receiving an assessment request from the user device, wherein the assessment request corresponds to accessing at least one historical risk assessment report for a timeframe amongst one or more risk assessment reports stored in the database. Moreover, the method includes retrieving, from the database, the at least one historical risk assessment report having the risk assessment timestamp within the timeframe. Furthermore, the method includes transmitting the at least one historical risk assessment report to the user device.

In some aspects of the present disclosure, the method further includes rendering, through the user device, one or more demonstration elements for each neuro-ophthalmic test from the one or more neuro-ophthalmic tests.

According to another embodiment, a system to perform neuro-ophthalmic risk assessment(s) of a user is presented. The system includes a database and a data processing circuitry communicatively coupled to the database. The data processing circuitry is configured to receive at least one facial image of the user from a user device. The data processing circuitry is further configured to determine parametric value(s) associated with at least one facial feature of the user, based on the at least one facial image through one or more neuro-ophthalmic tests. Furthermore, the data processing circuitry is configured to a measure of risk for the user based on the one or more parametric values using one or more Machine Learning (ML) models. The measure of risk indicates a level of neuro-ophthalmic risk associated with the user.

According to yet another embodiment, a computer-program product for neuro-ophthalmic risk assessment of a user is presented. The computer program product comprises computer-executable instructions that are stored on a non-transitory computer-readable medium and that, when executed by a data processing circuitry performs operations. The operations include receiving at least one facial image of the user from a user device. The operations further include determining parametric value(s) associated with at least one facial feature of the user, based on the at least one facial image through one or more neuro-ophthalmic tests. Furthermore, the operations include determining a measure of risk for the user based on the one or parametric values using one or more Machine Learning (ML) models. The measure of risk indicates a level of neuro-ophthalmic risk associated with the user.

### BRIEF DESCRIPTION OF DRAWINGS

Various embodiments disclosed herein will become better understood from the following detailed description when read with the accompanying drawings. The accompanying drawings constitute a part of the present disclosure and illustrate certain non-limiting embodiments of inventive concepts. Further, components and elements shown in the drawings are not necessarily to scale, emphasis instead being placed upon clearly illustrating the principles of the present disclosure. For the purpose of consistency and ease of understanding, similar components and elements are annotated by reference numerals in the example drawings. In the drawings:

FIG. 1 presents a block diagram that illustrates example components of a system to perform neuro-ophthalmic risk assessment(s) of a user, in accordance with an example aspect of the present disclosure.

FIG. 2 presents a block diagram that illustrates example components of a user device, in accordance with an embodiment of the present disclosure.

FIG. 3 presents a block diagram depicting example components of a data processing server, in accordance with an example embodiment of the present disclosure.

FIG. 4 presents a block diagram depicting example components of Machine Learning (ML) model(s), in accordance with an example embodiment of the present disclosure.

FIGS. 5(A)-5(F) illustrate Graphical User interfaces (GUIs) presented through the user device corresponding to the neuro-ophthalmic risk assessment of the user, in accordance with an example aspect of the present disclosure.

FIGS. 6(A)-6(E) illustrate process flow diagrams depicting steps performed by the system for various neuro-ophthalmic tests, in accordance with an example aspect of the present disclosure.

FIG. 7 is a flow chart that depicts a method for the neuro-ophthalmic risk assessment(s) of the user, in accordance with an example aspect of the present disclosure.

FIG. 8 is a flow chart that depicts a process for rendering a risk assessment report of the user, in accordance with an example embodiment of the present disclosure.

### DETAILED DESCRIPTION

Inventive concepts of the present disclosure will now be described more fully hereinafter with reference to the accompanying drawings, in which examples of one or more embodiments of inventive concepts are shown. Inventive concepts may, however, be embodied in different forms and should not be construed as limited to the embodiments set forth herein. Further, the one or more embodiments disclosed herein are provided to describe the inventive concept thoroughly and completely, and to fully convey the scope of each of the present inventive concepts to those skilled in the art. Furthermore, it should be noted that the embodiments disclosed herein are not mutually exclusive concepts. Accordingly, one or more components from one embodiment may be tacitly assumed to be present or used in any other embodiment.

The following description presents various embodiments of the present disclosure. The embodiments disclosed herein are presented as teaching examples and are not to be construed as limiting the scope of the present disclosure. The present disclosure should in no way be limited to the illustrative implementations, drawings, and techniques illustrated below, including the example design and implementation illustrated and described herein, but may be modified, omitted, or expanded upon without departing from the scope of the present disclosure.

The following description contains specific information pertaining to embodiments in the present disclosure. The detailed description uses the phrases “in some embodiments” which may each refer to one or more or all of the same or different embodiments. The term “some” as used herein is defined as “one, or more than one, or all.” Accordingly, the terms “one,” “more than one,” “more than one, but not all” or “all” would all fall under the definition of “some.” In view of the same, the terms, for example, “in an embodiment” refers to one embodiment and the term, for example, “in one or more embodiments” refers to “at least one embodiment, or more than one embodiment, or all embodiments.”

The term “comprising,” when utilized, means “including, but not necessarily limited to;” it specifically indicates open-ended inclusion in the so-described one or more listed features, elements in a combination, unless otherwise stated with limiting language. Furthermore, to the extent that the terms “includes,” “has,” “have,” “contains,” and other similar words are used in either the detailed description, such terms are intended to be inclusive in a manner similar to the term “comprising.”

In the following description, for the purposes of explanation, various specific details are set forth to provide a thorough understanding of embodiments of the present disclosure. It will be apparent, however, that embodiments of the present disclosure may be practiced without these specific details. Several features described hereafter can each be used independently of one another or with any combination of other features.

The description provided herein discloses example embodiments only and is not intended to limit the scope, applicability, or configuration of the present disclosure. Rather, the foregoing description of the example embodiments will provide those skilled in the art with an enabling description for implementing any of the example embodiments. Specific details are given in the following description to provide a thorough understanding of the embodiments. However, it may be understood by one of the ordinary skilled in the art that the embodiments disclosed herein may be practiced without these specific details.

The terminology used herein is for the purpose of describing particular embodiments only and is not intended to be limiting of the disclosure. As used herein the description, the singular forms “a”, “an”, and “the” include plural forms unless the context indicates otherwise.

The terminology and structure employed herein are for describing, teaching, and illuminating some embodiments and their specific features and elements and do not limit, restrict, or reduce the scope of the present disclosure. Accordingly, unless otherwise defined, all terms, and especially any technical and/or scientific terms, used herein may be taken to have the same meaning as commonly understood by one having ordinary skill in the art.

Various embodiments of the present disclosure relate to a system and a method for neuro-ophthalmic risk assessment of a user. The present system digitizes clinically validated ocular tests to track real-time deficiencies in eye movement and provide athletes with a concussion risk assessment. Using eye tracking and gaze estimation, the system collects parametric value(s) from tests assessing accommodative sufficiency, vestibular ocular reflex, saccadic eye movement, smooth pursuit eye movement, and optokinetic nystagmus. These tests are uniquely adapted from their clinical equivalents so as to be tested for on a computer application with greater precision and broader scope than visible by the naked eye. The novel combination of tests accounts for the ocular damages post-concussion and addresses greater accuracy, accessibility, and time efficiency for a user with a suspected head injury of the user.

Some embodiments of the present disclosure relate to use of Machine Learning (ML) model(s) specifically trained to classify data acquired from facial image(s) of the user to identify oculomotor impairment(s) and report the oculomotor impairment in significantly less time than what is observed through contemporary approaches. Specifically, the present disclosure relates to a use of a first ML model configured to determine a user feature metrics corresponding to the neuro-ophthalmic tests based on the parametric value(s), and a second ML model configured to compare the user feature metrics with a baseline feature metrics to determine a risk score(s) corresponding to a set of oculomotor impairments through neuro-ophthalmic test(s). The first ML model, in some cases, is Convolution Neural network (CNN) trained using baseline data comprising images of the user without any oculomotor impairment and multiple images related to various types of oculomotor impairments (set up as categories of classification of the ML model) to derive the feature metrics from facial images of the user pertaining to the neuro-ophthalmic test(s).

Some embodiments of the present disclosure relate to combination of oculomotor results derived through various neuro-ophthalmic tests assessing accommodative sufficiency, vestibular ocular reflex, saccadic eye movement, smooth pursuit eye movement, and optokinetic nystagmus. This combination gives rise to the risk score that enables the system to assign a risk category from multiple impairment categories to the user, such that the medical state of the user can be identified without a significant delay. This further helps in facilitating the user with accurate emergency aid for immediate treatment of the user in case of fatal injuries, and thus can be life saver for various professionals (such as athletes, boxers, sportsmen etc.).

Some other embodiments of the present disclosure relate to tracking of personal health state(s), that can help the user to keep a track of health through historical risk assessment reports generated by the system and saved with a timestamp. Some other embodiments of the present disclosure relate to two modes of operation of the system i.e., self-assessment and guided (or assisted) assessment. The self-assessment feature of the system enables the user to perform neuro-ophthalmic risk assessment on own, whereas the guided assessment feature facilitates another user to assist in neuro-ophthalmic risk assessment of the user. The guided (or assisted) assessment feature enables neuro-ophthalmic risk assessment of a user with fatal injuries (i.e., critical condition) to be performed by the system.

The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

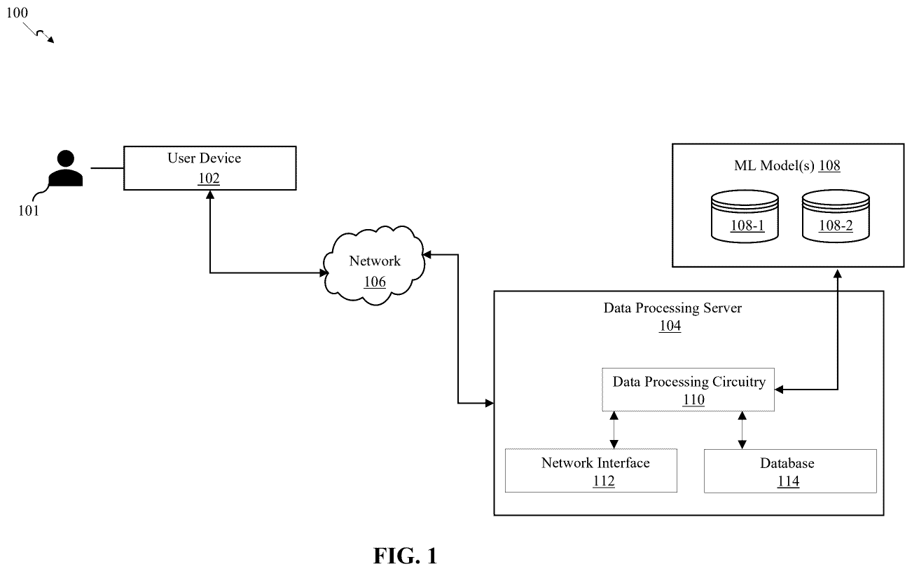

Embodiments of the present disclosure will be described below in detail with reference to the accompanying drawings. FIG. 1 through FIG. 8, discussed below, and the embodiments used to describe the principles of the present disclosure are by way of illustration only and should not be construed in any way to limit the scope of the present disclosure. Those skilled in the art will understand that the principles of the present disclosure may be implemented in any suitably arranged system or device.

Various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

FIG. 1 presents a block diagram that illustrates example components of a system 100 to perform neuro-ophthalmic risk assessment of a user 101, in accordance with an example aspect of the present disclosure. The system 100 may include a user device 102 and a data processing server 104 coupled to each other by way of a network 106. The data processing server 104 is supported by Machine Learning (ML) model(s) 108.

The user device 102 may be capable of communicating data and/or instructions with the data processing server 104 through the network 106. The user device 102 may have an electronic application (i.e., app) installed, that may enable the user 101 to interact with the data processing server 104. The electronic application may be hosted by the data processing server 104, such that an application interface of the electronic application (i.e., displayed on the user device 102) may enable the user 101 to provide input(s) and receive output(s) corresponding to neuro-ophthalmic risk assessment.

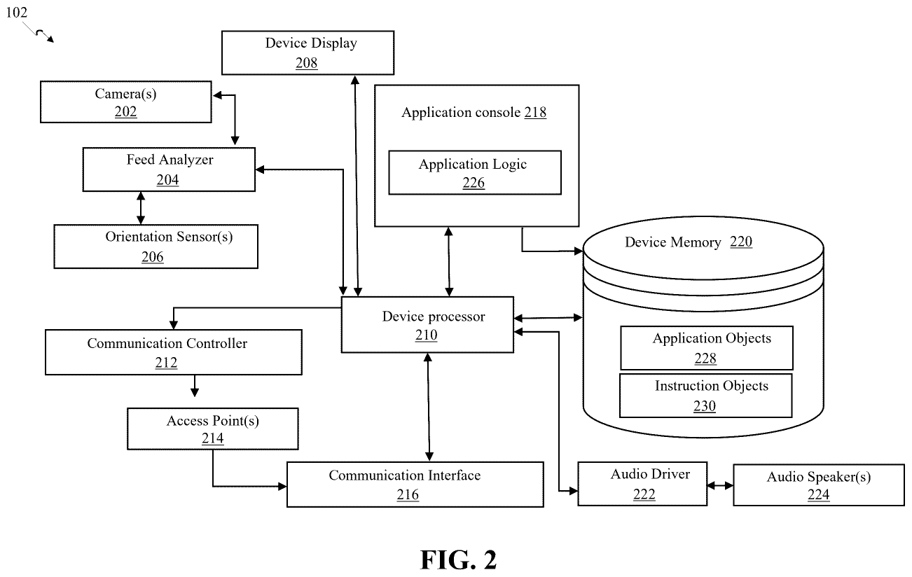

More particularly, the electronic application may be capable of capturing facial images of the user 101 (using camera(s) 202 of the user device 102 shown later in FIG. 2) and share the facial images of the user 101 with the data processing server 104. The electronic application may further provide orientation output(s) to the user 101, which may enable the user 101 to capture the facial image(s) in a pre-defined images particular to the ML model(s) 108. Furthermore, the electronic application enables the user 101 to provide baseline data comprising images of the user 101 without any impairment which is utilized by the system 100 for training of the ML model 108 and supporting the data processing server 104 in concession risk assessment.

Examples of the user device 102 may include, but are not limited to portable handheld electronic devices such as a mobile phone, a tablet, a laptop, a smart watch etc., or fixed electronic devices such as a desktop computer, computing devices, etc. Aspects of the present disclosure are intended to include or otherwise cover any type of user devices, available now or later developed through advancement in technology, as the user device 102, without deviating from the scope of the present disclosure.

The data processing server 104 may be configured to perform data processing operations and/or data storage operations related to neuro-ophthalmic risk assessment of the user 101. More particularly, the data processing server 104 may be configured to receive facial image(s) of the user 101 from the user device 102 and determine parametric value(s). The parametric value(s) may be associated with facial feature(s) of the user derived by analyzing oculomotor reflexes of the user 101 captured in the facial image(s). In some aspects of the present disclosure, the facial feature(s) may be associated with tracking a movement of eyes of the user and estimating a gaze of the eyes of the user.

The data processing server 104 may further be configured to identify impairment level(s) for impairment(s) based on the parametric value(s) using the ML model(s) 108. In some aspects of the present disclosure, the data processing server 104 may determine a risk score(s) corresponding to neuro-ophthalmic test(s) using the parametric value(s) through the ML model(s) 108. In some aspects of the present disclosure, the ML model(s) 108 may support the data processing server 104 by determining the user feature metrics corresponding to the neuro-ophthalmic test(s) based on the parametric value(s). The ML model(s) 108 may further support the data processing server 104 by comparing the user feature metrics with the baseline feature metrics to determine the risk score(s).

Based on the risk score(s), the data processing server 104 may identify oculomotor impairment(s) (if any) for the user 101. In a scenario, when the data processing server 104 determines oculomotor impairment(s) for the user 101, the data processing server 104 may generate a risk assessment report. In another scenario, when the data processing server 104 determines no oculomotor impairment for the user 101, the data processing server 104 may generate a non-impaired report for the user 101.

Furthermore, the data processing server 104 may be configured to determine a measure of risk for the user 101. The measure of risk may indicate a level of neuro-ophthalmic risk associated with the user 101. In some aspects of the present disclosure, the measure of risk may be associated with a risk category from multiple risk categories. The data processing server 104 may assign the risk category to the user 101 based on the impairment level(s). In some aspects of the present disclosure, the risk category may be one of “no risk category”, “low risk category”, moderate risk category”, and “high risk category”. Aspects of the present disclosure are intended to include or otherwise cover any type of classification and/or categorization of the user 101 based on the impairment level(s), without deviating from the scope of the present disclosure.

The data processing server 104 may also be configured to render at least one of, the measure of risk, the risk category, the risk score(s), and one of a risk assessment report or a non-impaired report for the user 101 to the user device 102. In another scenario, when the data processing server 104 receives an assessment request to retrieve historical data corresponding to the user's historical oculomotor state, the data processing server 104 may identify a timeframe associated with the assessment request, retrieve historical information of the user's oculomotor state corresponding to the timeframe, and render the historical information of the user 101 through the user device 102.

The data processing server 104 may be a network of computers, a software framework, or a combination thereof, that may provide a generalized approach to create a server implementation. Examples of the data processing server 104 may include, but are not limited to, personal computers, laptops, mini-computers, mainframe computers, any non-transient and tangible machine that can execute a machine-readable code, cloud-based servers, distributed server networks, or a network of computer systems. The data processing server 104 may be realized through various web-based technologies such as, but not limited to, a Java web-framework, a .NET framework, a personal home page (PHP) framework, or any web-application framework.

The data processing server 104 may include data processing circuitry 110, a network interface 112, and a database 114. The data processing circuitry 110 may include processor(s) configured with suitable logic, instructions, circuitry, interfaces, and/or codes for executing various operations performed by the data processing server 104 for neuro-ophthalmic risk assessment of the user 101. Examples of the data processing circuitry 110 may include, but are not limited to, an Application Specific Integrated Chip (ASIC) processor, a RISC processor, a CISC processor, a Field Programmable Gate Array (FPGA), and the like.

The network interface 112 may be configured to enable the data processing server 104 to communicate with the user device 102 via the network 106. Examples of the network interface 112 may include, but are not limited to, a MODEM, a network interface such as an Ethernet card, a communication port, and/or a Personal Computer Memory Card International Association (PCMCIA) slot and card, an antenna, a radio frequency (RF) transceiver, amplifier(s), a tuner, oscillator(s), a digital signal processor, a coder-decoder (CODEC) chipset, a Subscriber Identity Module (SIM) card, and a local buffer circuit. It will be apparent to a person of ordinary skill in the art that the network interface 112 may include any device and/or apparatus capable of providing wireless or wired communications between the data processing server 104 and the user device 102.

The database 114 may be configured to store the logic, instructions, circuitry, interfaces, and/or codes of the data processing circuitry 110 for executing various operations of the data processing server 104 related to the neuro-ophthalmic risk assessment of the user 101. Aspects of the present disclosure are intended to include and/or otherwise cover any type of the data associated with the data processing server 104, without deviating from the scope of the present disclosure. Examples of the database 114 may include but are not limited to, a ROM, a RAM, a flash memory, a removable storage drive, a HDD, a solid-state memory, a magnetic storage drive, a PROM, an EPROM, and/or an EEPROM.

The data processing server 104 may be supported by the ML model(s) 108 in performing Artificial Intelligence based tasks specific to feature extraction and data classification for neuro-ophthalmic risk assessment of the user based on analysis of the facial image(s). In some aspects of the present disclosure, the ML model(s) 108 may include a first ML model 108-1 and a second ML model 108-2. The first ML model 108-1 may be configured to determine a user feature metrics corresponding to the neuro-ophthalmic test(s) based on the parametric value(s), and the second ML model 108-2 may be configured to compare the user feature metrics with the baseline feature metrics to determine the risk score(s). Specifically, the first ML models 108-1 may be trained with baseline data comprising facial images of the user 101 without any oculomotor impairment and facial images corresponding to oculomotor impairments. The first ML model 108-1 may support the data processing server 104 in deriving facial feature(s) pertaining to the neuro-ophthalmic test(s) and determine the user feature metrics based on the facial feature(s). In some aspects of the present disclosure, the second ML model 108-2 may support the data processing server 104 in identifying oculomotor impairment(s) and severity of the oculomotor impairment(s) by analyzing the facial feature(s). Moreover, the second ML model 108-2 may also support the data processing server 104 in determining the measure of risk and/or assigning the risk category to the user 101.

In some other aspects of the present disclosure, the functionality derived through the first ML model 108-1 and the second ML model 108-2 can be achieved using a single ML model configured to determine the user feature metrics and determine the risk category based on the comparison of the feature metrics with the baseline feature metrics.

In some aspects of the present disclosure, the ML model(s) 108 may be hosted by external datacenter(s). The external datacenter(s) (not shown) may include suitable logic, circuitry, and/or code(s) to facilitate the ML model(s) 108 to store data and perform computational tasks for supporting the data processing server 104. Examples of the external data center(s) may include, but are not limited to Oracle Database, Amazon Web Services (AWS) Database, and the like.

The network 106 may include suitable logic, circuitry, and interfaces that may be configured to provide several network ports and several communication channels for transmission and reception of data related to operations of various entities of the system 100. Each network port may correspond to a virtual address (or a physical machine address) for transmission and reception of the communication data. For example, the virtual address may be an Internet Protocol Version 4 (IPV4) (or an IPV6 address) and the physical address may be a Media Access Control (MAC) address. The network 106 may be associated with an application layer for implementation of communication protocols based on communication requests from the various entities of the system 100. The communication data may be transmitted or received via the communication protocols. Examples of the communication protocols may include, but are not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof. In some aspects of the present disclosure, the communication data may be transmitted or received via at least one communication channel of several communication channels in the network 106. The communication channels may include, but are not limited to, a wireless channel, a wired channel, a combination of wireless and wired channel thereof. The wireless or wired channel may be associated with a data standard which may be defined by one of a Local Area Network (LAN), a Personal Area Network (PAN), a Wireless Local Area Network (WLAN), a Wireless Sensor Network (WSN), Wireless Area Network (WAN), Wireless Wide Area Network (WWAN), a metropolitan area network (MAN), a satellite network, the Internet, an optical fiber network, a coaxial cable network, an infrared (IR) network, a radio frequency (RF) network, and a combination thereof. Aspects of the present disclosure are intended to include or otherwise cover any type of communication channel, including known, related art, and/or later developed technologies.

Although FIG. 1 illustrates one example of the system 100, various changes may be made to FIG. 1. For example, the system 100 may include any number of functional nodes, servers, and/or user devices in any suitable arrangement, without deviating from the scope of the present disclosure. Further, various components in FIG. 1 may be combined, further subdivided, or omitted and additional components may be added according to particular needs.

FIG. 2 presents a block diagram that illustrates example components of the user device 102, in accordance with an embodiment of the present disclosure. The user device 102 may include camera(s) 202, a feed analyzer 204, orientation sensor(s) 206, a user interface 208, a device processor 210, a communication controller 212, access point(s) 214, a communication interface 216, an application console 218 comprising an application logic 226, a device memory 220 comprising application objects 228 and instruction objects 230, an audio driver 222, and audio speaker(s) 224.

The camera(s) 202 may facilitate the user 101 to capture the facial image(s). Examples of the camera(s) 202 may include, but not limited to a fixed camera module, a Pan-Tilt-Zoom camera, a mobile camera, a double-lens camera, and the like. Aspects of the present disclosure are intended to include any camera module present in the electronic devices present now or in later developed technologies capable of capturing the facial image(s) of the user 101 as the camera(s) 202.

The orientation sensor(s) 206 may include electronic sensor(s) configured to determine parameters related to the orientation of the user device 102. Examples of the orientation sensor(s) 206 may include an accelerometer sensor, a gyroscope sensor, a magnetometer sensor, and the like. Aspects of the present disclosure are intended to include or otherwise cover any type of electronic sensor capable of determining the orientation of the user device 102 as the orientation sensor(s) 206, without deviating from the scope of the present disclosure.

The feed analyzer 204 may be configured to assess input(s) received from the orientation sensor(s) and assist in capturing the facial image(s) using the camera(s) 202. The feed analyzer 204 may further be communicatively coupled with the device processor 210 that assists the feed analyzer to control the camera(s) capture facial image(s) in a predefined orientation specific to the ML model(s) 108.

The user interface 208 may include an input interface (not shown) for receiving input(s) from the user 101. Examples of the input interface may include, but are not limited to, a touch interface, a mouse, a keyboard, a motion recognition unit, a gesture recognition unit, a voice recognition unit, or the like. Aspects of the present disclosure are intended to include or otherwise cover any type of the input interface including known, related art, and/or later developed technologies without deviating from the scope of the present disclosure. The user interface 208 may further include an output interface for rendering output(s) to the user 101. Examples of the output interface may include, but are not limited to, a digital display, an analog display, a touch screen display, a graphical user interface, a website, a webpage, a keyboard, a mouse, a light pen, an appearance of a desktop, and/or illuminated characters. Aspects of the present disclosure are intended to include or otherwise cover any type of the output interface including known, related art, and/or later developed technologies without deviating from the scope of the present disclosure.

The application console 218 may be configured as a computer-executable application, to be executed by the user device 102. The application console 218 may include suitable logic, instructions, and/or codes for executing multiple operations of the system 100 and may be controlled (or hosted) by the data processing server 104. The computer executable application(s) may be stored in the device memory 220. In some aspects of the present disclosure, the application console 218 may include an application logic 226, that may include logic, codes, and/or circuitry to control the display through the output interface. More particularly, the application logic may be shared with the device processor 210 that controls output(s) rendered through the output interface.

The device processor 210 may include suitable logic, instructions, circuitry, interfaces, and/or codes for executing various operations associated with the user device 102. In some aspects of the present disclosure, the device processor 210 may utilize processor(s) such as Arduino or raspberry pi and/or the like. Further, the device processor 210 may be configured to control operation(s) executed by the user device 400 in response to the input received at the user interface 208 from the user 101. Examples of the device processor 210 may include, but are not limited to, an application-specific integrated circuit (ASIC) processor, a reduced instruction set computing (RISC) processor, a complex instruction set computing (CISC) processor, a field-programmable gate array (FPGA), a Programmable Logic Control unit (PLC), and the like. Aspects of the present disclosure are intended to include or otherwise cover any type of the device processor 210 including known, related art, and/or later developed processing units, without deviating from the scope of the present disclosure.

The device memory 220 may be configured to store logic, instructions, circuitry, interfaces, and/or codes of various other components of the user device 102. Examples of the device memory 220 may include, but are not limited to, a Read-Only Memory (ROM), a Random-Access Memory (RAM), a flash memory, a removable storage drive, a hard disk drive (HDD), a solid-state memory, a magnetic storage drive, a Programmable Read Only Memory (PROM), an Erasable PROM (EPROM), and/or an Electrically EPROM (EEPROM). Aspects of the present disclosure are intended to include or otherwise cover any type of the device memory 220 including known, related art, and/or later developed memories, without deviating from the scope of the present disclosure. In some aspects of the present disclosure, the device memory 220 may store application objects 228 specific to the computer-executable application running through the application console 218. The device memory 220 may further store instruction objects for operations of various components of the user device 102.

The communication controller 212 may include circuitry to enable and/or control the access point(s) 214. The access point(s) 214 may generate wireless communication signals that facilitate the communication interface 216 to communicatively couple with network 106.

The communication interface 216 may be configured to enable the user device 102 to communicate with the data processing server 104 over the network 106. Examples of the communication interface 216 may include, but are not limited to, a modem, a network interface such as an Ethernet card, a communication port, and/or a Personal Computer Memory Card International Association (PCMCIA) slot and card, an antenna, a radio frequency (RF) transceiver, amplifier(s), a tuner, oscillator(s), a digital signal processor, a coder-decoder (CODEC) chipset, a Subscriber Identity Module (SIM) card, and a local buffer circuit. It will be apparent to a person of ordinary skill in the art that the communication interface 216 may include any device and/or apparatus capable of providing wireless or wired communication between the user device 102 and the data processing server 104.

The audio driver 222 may be configured to control the audio speaker(s) 224 to produce sound(s) specific to the neuro-ophthalmic test(s). Preferably, the audio driver 222 may control output through the audio speaker(s) 224 to generate audible metronome paced rhythm that facilitates the user 101 to capture facial image(s) at specific angles pertaining to the neuro-ophthalmic test(s).

Although FIG. 2 illustrates one example of the user device 102, various changes may be made to FIG. 2. For example, the user device 102 may include any number of functional components such as those presented through FIG. 2 in any suitable arrangement, without deviating from the scope of the present disclosure. Further, various components in FIG. 2 may be combined, further subdivided, or omitted and additional components may be added according to particular needs.

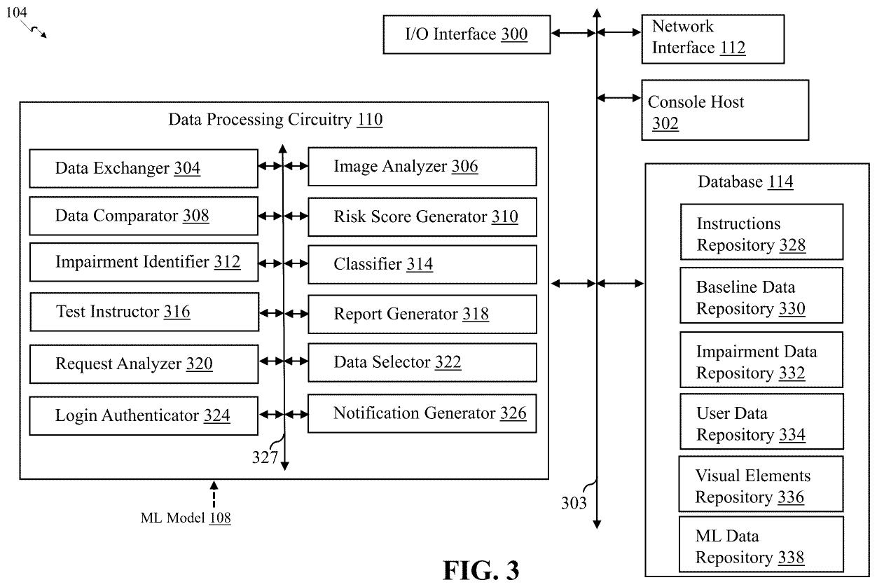

FIG. 3 illustrates a block diagram depicting example components of the data processing server 104, in accordance with an example embodiment of the present disclosure. The data processing server 104 may be configured to perform data processing task(s) and data storage task(s) for neuro-ophthalmic risk assessment of the user 101. According to the example embodiment as presented through FIG. 3, the data processing server 104 may include the data processing circuitry 110, the database 114, the network interface 112, an input-output (I/O) interface 300, and a console host 302, coupled to each other by way of a first communication bus 303.

The I/O interface 300 may include suitable logic, circuitry, interfaces, and/or codes that may be configured to receive input(s) and render output(s) by or from the data processing server 104, respectively. The input(s) may correspond to operation(s) and configuration(s) of various components of the data processing server 104. The output(s) may correspond to an operational status of various components of the data processing server 104.

The console host 302 may include suitable logic, circuitry, interfaces, and/or codes that may be configured for executing various operations of the electronic application on the user device 102, by way of which the user 101 can trigger the data processing server 104 for neuro-ophthalmic risk assessment and/or retrieving the historical information of neuro-ophthalmic risk assessment(s) of the user 101. In some other aspects of the present disclosure, the console host 302 may further provide Graphical User Interfaces (GUIs) to enable the data processing server 104 for user interaction.

The data processing circuitry 110 may include a data exchanger 304, an image analyzer 306, a data comparator 308, a risk score generator 310, an impairment identifier 312, a classifier 314, a test instructor 316, a report generator 318, a request analyzer 320, a data selector 322, a login authenticator 324, and a notification generator 326, communicatively coupled to each other by way of a second communication bus 327.

The login authenticator 324 may be configured to receive user registration data from the user device 102 at a first instance of account signup. The user registration data may include personal identifier containing identity information of the user of the user device. In some aspects of the present disclosure, the user registration data may also include biometric data of the user such as, but not limited to, fingerprints, iris scans, images, voice samples, and the like associated with the user. Aspects of the present disclosure are intended to include or otherwise cover any type of biometric data of the plurality of users without deviating from the spirit and scope of the present disclosure. Moreover, the login authenticator 324 may generate a user's account for the user 101 to enable the user for the neuro-ophthalmic risk assessment. The login authenticator 324 may also be configured to verify the registration data at each instance of login to enable the user device 102 for utilizing the services of the data processing server 104 towards neuro-ophthalmic risk assessment.

In some other aspects of the present disclosure, the login authenticator 324 may enable the user 101 to set a password for logging-in to the system 100. In such a scenario, the login authenticator 324 may be configured to verify a password entered by the user for logging-in to the system 100 by comparing the password entered by the user with the set password. In a scenario, when the password entered by the user is verified, the login authenticator 324 enables the user 101 to utilize the service(s) of the data processing server 104 towards the neuro-ophthalmic risk assessment. In a scenario, when the password entered by the user is not verified, the login authenticator 324 may generate a login error signal to enable a login error to be displayed on the user device 102 to notify the user 101 for denied usage of service(s) of the data processing server 104 towards the neuro-ophthalmic risk assessment.

The data exchanger 304 may be configured to enable exchange of data and/or instruction(s) between the various other components of the data processing circuitry 110 and the database 114, the ML model(s) 108, and the user device 102. Particularly, the data exchanger 304 may be configured to receive the facial image(s) of the user 101 from the user device 102. The data exchanger 304 may further be configured to share the facial image(s) of the user 101 with the image analyzer 306. The image analyzer 306 may be configured to determine the parametric value(s) associated the facial feature(s) of the user. In some aspects of the present disclosure, the facial feature(s) may correspond to analysis of oculomotor reflexes of the user 101 from the facial image(s). In some aspects of the present disclosure, the facial feature(s) may be associated with tracking of the movement of eyes of the user 101 and estimating the gaze of the eyes of the user 101.

In some aspects of the present disclosure, the image analyzer 306 may identify pixel(s) from each of the facial image(s) comprising facial feature(s) corresponding to the neuro-ophthalmic test(s). The image analyzer 306 may further be configured share the facial feature(s) with the data comparator 308. The data comparator 308 may be configured to compare the facial feature(s) with a set of baseline feature(s) corresponding to the neuro-ophthalmic test(s) to extract the parametric value(s). Moreover, the image analyzer 306 may be configured to determine the user feature metrics from the facial feature(s) based on the parametric value(s). The user feature metrics may correspond to the neuro-ophthalmic test(s). The image analyzer 306 and the data comparator 308 may be supported by the ML model(s) 108 for comparing the feature(s) extracted from the facial image(s) with the set of baseline feature(s) and determining the user feature metrics corresponding to the neuro-ophthalmic test(s).

In some aspects of the present disclosure, the neuro-ophthalmic test(s) comprises an accommodation test to measure an eye focus adjustment of the user 101, a Vestibulo-ocular Reflex (VOR) test to measure a stability of the eyes of the user 101, a saccadic eye movement test to evaluate a rapid gaze shift of the user 101, a smooth pursuit test to analyze continuous object tracking by the user 101, and an optokinetic nystagmus test to analyze response to a succession of moving stimuli on the user 101.

The risk score generator 310 may be configured to determine a risk score(s) for the user 101 corresponding to the neuro-ophthalmic test(s) using the parametric value(s). Particularly, the risk score generator 310 may be configured to compare the user feature metrics with the baseline feature metrics to determine the risk score(s). The risk score generator 310 may also be supported by the ML model(s) 108 for determining the risk score(s) for the user. In some aspects of the present disclosure, the ML model(s) 108 may support the risk score generator 310 in determining the risk score(s) by comparing the user feature metrics with the baseline feature metrics. The risk score generator 310 may further be configured to share the risk score(s) with the data comparator 308.

In some aspects of the present disclosure, the data comparator 308 may also be configured to compare the risk score(s) with a non-impaired range. The non-impaired range may include a range of threshold value for each risk score of the risk score(s) corresponding to a neuro-ophthalmic test. The data comparator 308 may further determine whether risk score(s) are within the corresponding non-impaired range. In a scenario, when the data comparator 308 determines that all the risk scores in the risk score(s) are in the non-impaired range, the data comparator 308 may generate a no-neuro-ophthalmic trigger for the report generator 318, which may enable the report generator 318 to generate the non-impaired report for the user 101. The non-impaired report of the user 101 may include the facial image(s), the feature metrics, and the parametric value(s) derived from the facial image(s). In some aspects of the present disclosure, the contents of the non-impaired report may be added to the baseline feature metrics.

In another scenario, when the data comparator 308 determines that risk score(s) are not within the non-impaired range, the data comparator 308 may generate a neuro-ophthalmic trigger for the impairment identifier 312. Based on the neuro-ophthalmic trigger, the impairment identifier 312 may identify oculomotor impairment(s) from a plurality of predefined oculomotor impairments, associated with the neuro-ophthalmic test(s) corresponding to the identified risk score(s) higher than the corresponding threshold value(s). For both the scenarios, the classifier 314 may be configured to determine the measure of risk for the user 101. Particularly, the classifier 314, based on the parametric value(s) may be configured to identify impairment level(s) for impairment(s) associated with the neuro-ophthalmic test(s). The classifier 314 may also be supported by the ML model(s) 108 for identifying the impairment level(s). Moreover, the classifier 314 may be configured to determine the measure of risk for the user 101 based on the impairment level(s). The measure of risk may indicate the level of neuro-ophthalmic risk associated with the user 101.

In the scenario, when oculomotor impairment(s) are identified for the user 101, the classifier 314 may be configured to determine a risk category (from multiple risk categories) for the user based on the measure of risk. In some aspects of the present disclosure, the classifier 314 may be configured to determine data elements associated with information of the oculomotor impairment(s) based on the comparison of the facial feature(s) derived from the facial image(s) with the set of baseline features. The classifier 314 may further instruct the report generator 318 to generate a risk assessment report based on data elements. The data exchanger 304 may further be configured to store the risk assessment report with a risk assessment timestamp in the database 114. The data exchanger 304 may further transmit the risk assessment report to the user device 102, to be rendered to the user 101.

In some aspects of the present disclosure, the test instructor 316 may be configured to generate trigger signal(s) for the user device to facilitate the user in capturing the facial image(s) for the neuro-ophthalmic test(s) in a predefined orientation specific to the ML model(s) 108. The test instructor 316 may further be configured to render demonstration element(s) (e.g., audio elements, video elements, etc.) for each neuro-ophthalmic test from the neuro-ophthalmic test(s) through the user device 102. The demonstration element(s) may be stored in the database 114 and/or the device memory 220 (as application object(s) 228). In some other aspects of the present disclosure, the test instructor 316 may also be configured to receive a perspective input from the user device 102 for the neuro-ophthalmic test(s). The perspective input may correspond to an individual using the user device 102 for capturing the facial image(s) of the user 101. Specifically, the perspective input may correspond to an individual mode of operation (where the user 101 captures the facial image(s)) or an assisted mode of operation (where the user 101 is assisted to capture the facial image(s)). The demonstration element(s) may be different in both the modes of operation. The notification generator may be configured to generate notification(s) for the user 101 reflecting operation(s) and/or outcome(s) derived by the data processing circuitry 110.

In an example scenario, when an assessment request is received from the user device 102 for a historical event (e.g., past risk assessment(s) of the user 101) by the request analyzer 320, the request analyzer 320 may retrieve a timeframe for the assessment request. The data selector 322 may be configured to retrieve historical risk assessment report(s) having the risk assessment timestamp within the timeframe. Moreover, the data selector 322 may be configured to transmit the historical risk assessment report(s) to the user device 102.

Various components of the data processing circuitry 110 are presented to illustrate the functionality driven by the data processing server 104. It will be apparent to a person having ordinary skill in the art that various components in the data processing circuitry 110 are for illustrative purposes, and not limited to any specific combination of hardware circuitry and/or software.

The database 114 may be configured to store data of the data processing server 104 and/or the ML model(s) 108. In some aspects of the present disclosure, database 114 may be segregated into multiple repositories that may be configured to store a specific type of data. In the example embodiment as presented through FIG. 3, the database 114 may include an instructions repository 328, a baseline data repository 330, an impairment data repository 332, a user data repository 334, a visual elements repository 336, and a ML data repository 338.

The instructions repository 328 may be configured to store instructions for various components of the data processing server 104. The baseline data repository 330 may be configured to store the baseline data for various neuro-ophthalmic test(s). The baseline data may include feature(s), facial image(s), and parametric value(s) associated with an impairment-free state of the user(s) 101 of the system 100. The impairment data repository 332 may be configured to store impairment data of the users 101. The impairment data may include feature(s), facial image(s), and parametric value(s) associated with oculomotor impairment(s) from the predefined oculomotor impairments. The user data repository 334 may be configured to store data associated with registration and/or authentication of users 101 of the system 100. The visual elements repository 336 may be configured to store the visual element(s) corresponding to the neuro-ophthalmic test(s). The visual elements may be stored as image(s), video(s), audio(s) files in the visual elements repository 336, that may guide the user 101 to capture the facial image(s). The ML data repository 338 may be configured to retrieve data and/or instruction(s) from the ML model(s) 108 that may be utilized by various components of the data processing circuitry 110 for determination of oculomotor impairment(s) and assignment of the risk category to the user 101.

Various components of the database 114 are presented for illustration as per the functionality of the data processing server 104. It will be apparent to a person having ordinary skill in the art that various components in the database 114 are for illustrative purposes and the scope of the present disclosure is not limited by the specific repositories as presented herein through FIG. 3. The database 114 may include any count and/or type of data storage repositories, without deviating from the scope of the present disclosure.

Although FIG. 3 illustrates one example of the data processing server 104, various changes may be made to FIG. 3. For example, the data processing server 104 may include any number of functional components (similar to those presented in FIG. 3) in any suitable arrangement, without deviating from the scope of the present disclosure. Further, various components of the data processing server 104 (as presented in FIG. 3) may be combined, further subdivided, or omitted and additional components may be added according to particular needs.

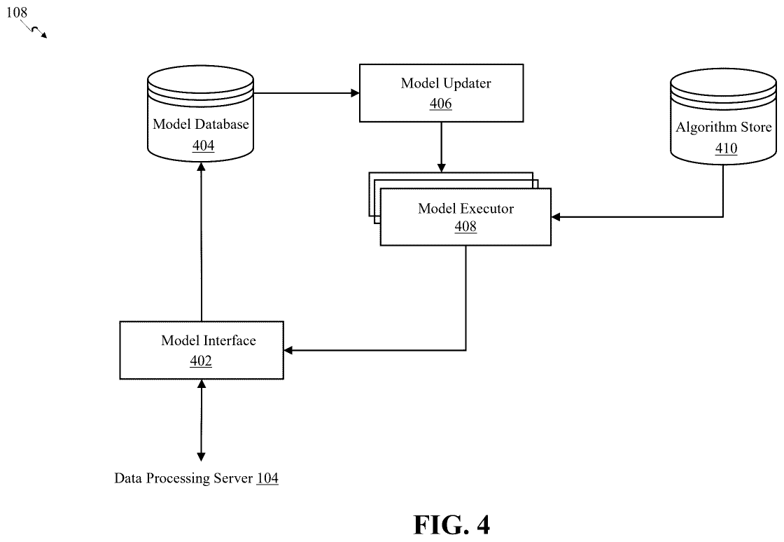

FIG. 4 presents a block diagram depicting example components of the Machine Learning (ML) model(s) 108 used to support the data processing server 104 in neuro-ophthalmic risk assessment of the user 101, in accordance with an example embodiment of the present disclosure. FIG. 4 illustrates one ML model(s) 108 as an example embodiment, which may be one of the first ML model 108-1 and/or the second ML model 108-2. The architecture of the ML model is generic and can be utilized for implementation of the first ML model 108-1 and the second ML model 108-2. FIG. 4 presents example components of the ML model(s) 108 for illustration of the functionality of the ML model(s) 108. It will be apparent to a person of ordinary skill in the art that the architecture of the ML model(s) 108 is not restricted to the components presented in FIG. 4 and is rather dependent on the scope of the present disclosure.

Each of the ML model(s) 108 (hereinafter interchangeably referred to and designated as ‘the ML model 108’) may include a model interface 402, a model database 404, a model updater 406, a model executor 408, and an algorithm store 410. In the example embodiment, the first and the second ML models 108 (as discussed in FIG. 1) may be designed in accordance with the ML model 108 as presented herein.

The model interface 402 may receive training data based on the functionality of various components of data processing circuitry 110. The model interface 402 may further send model-generated output(s) to the data processing server 104. Furthermore, the model interface 402 may receive feedback data from the data processing server 104 and may transmit the feedback data to model database 404. The model updater 406 may access the feedback data to update parameter(s) (such as weights, bias, number of layers, filters, pooling type etc.) of the model executor 408 for training specific to classification of the facial image(s) for neuro-ophthalmic risk assessment of the user. Specifically for the first ML model 108-1, the model updater 406 may update the parameters for the determination of the user feature metrics corresponding to the neuro-ophthalmic test(s) based on the parametric value(s). Moreover, for the second ML model 108-2, the model updater 406 may update the parameters for comparing the user feature metrics with the baseline feature metrics to determine the risk score(s). Additionally, the model interface 402 may receive instruction data from the data processing server 104 and may transmit classified information to the data processing server 104.

The model database 404 may store neural networks, weights of neurons for the neural networks, input data for the neural networks, output data from the neural networks, and the like. Additionally, the model database 404 may transmit a neural network from the stored neural networks to the model updater 406. The model database 404 may further send the feedback data to model updater 406. Based on the feedback data, the model updater 406 may update the parameters of ML model 108 for customized training specific to each functionality of the data processing server 104.

The model executor 408 may receive data from the model updater 406 that includes a customized training regimen specific to each object and the feedback data. Based on the data received from the model updater 406, the model executor 408 may retrieve ML algorithm(s) from the algorithm store 410 to perform operation(s) for the data processing server 104. Particularly, the model executor 408 may include an input neurons layer configured to receive data from the model interface 402, hidden neuron layer(s) configured to propagate the received data for classification, and an output neuron layer configured to depict an output in accordance with the functionality of the component(s) of the data processing server 104. Moreover, the model executor 408 is trained for specific task(s) to classify the input data to generate the output. Each neuron of the model executor 408 may be attached with a weight and a bias, that is determined via training of the ML model 108.

In some aspects of the present disclosure, the model executor 408 may be designed using field programmable gate array (FPGA) and/or application specific integrated chip (ASIC) programmed for a specific ML functionality to support the data processing server 104. It will be apparent to a person of ordinary skill in the art that the scope of the ML model(s) 108 is not limited only to use of any specific ML algorithm. Rather, the scope of the present disclosure is limited to the functionality of the data processing server 104 as presented in FIG. 3, that may be supported to utilize any ML model, existing or designed later in advancement of the technology, without deviating from the scope of the present disclosure.

In some aspects of the present disclosure, the model executor 408 may transmit indication of a ML algorithm to algorithm store 410. The algorithm store 410 may store multiple ML algorithms. Based on the indication, the algorithm store 410 may transmit the corresponding ML algorithm from the multiple ML algorithms to be used by the model executor 408 for neuro-ophthalmic risk assessment of the user 101.

Although FIG. 4 illustrates one example of the ML model 108, various changes may be made to FIG. 4. For example, the ML model(s) 108 may include any number of functional components (similar to those presented for the ML model 108 in FIG. 4) in any suitable arrangement, without deviating from the scope of the present disclosure. Further, various components of the ML model(s) 108 may be combined, further subdivided, or omitted and additional components may be added according to particular needs.

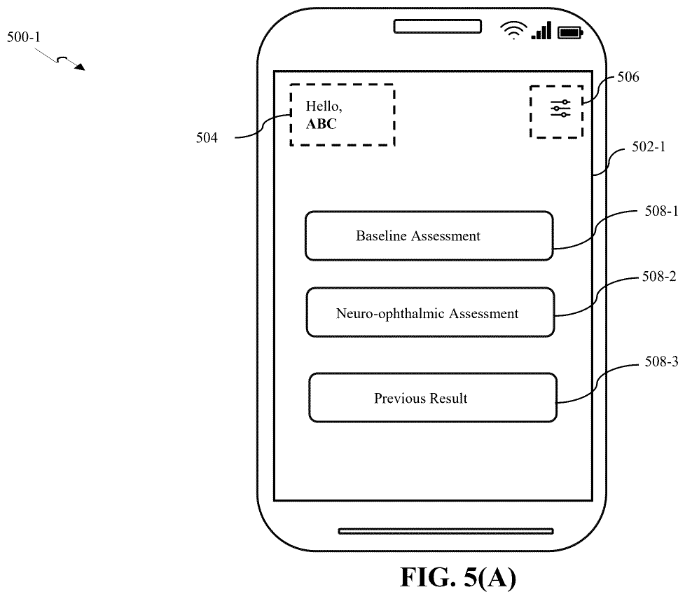

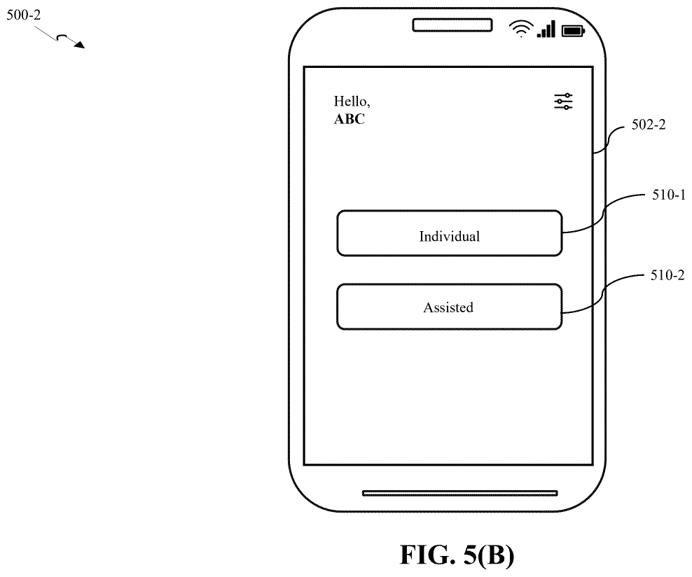

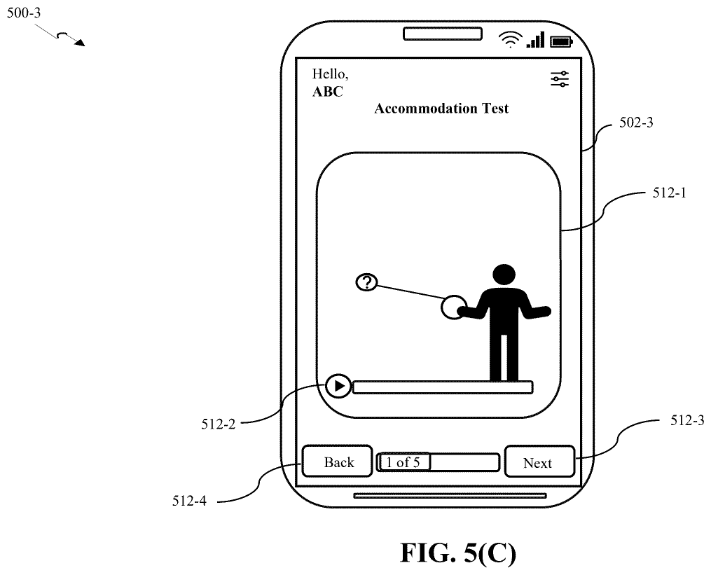

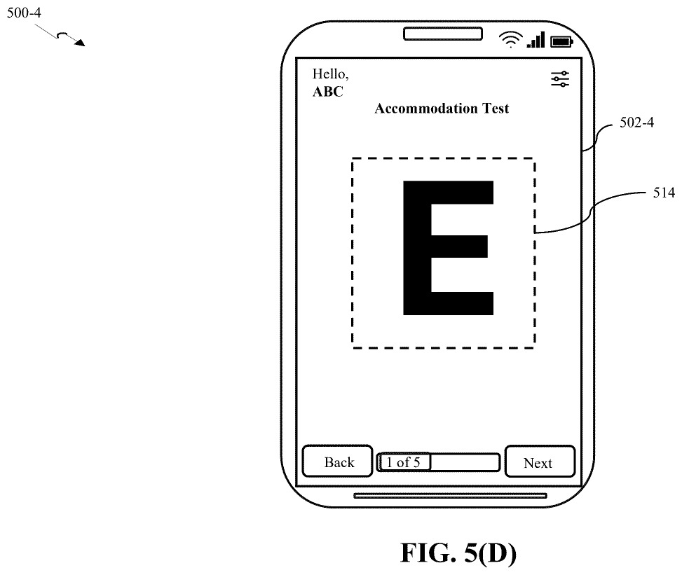

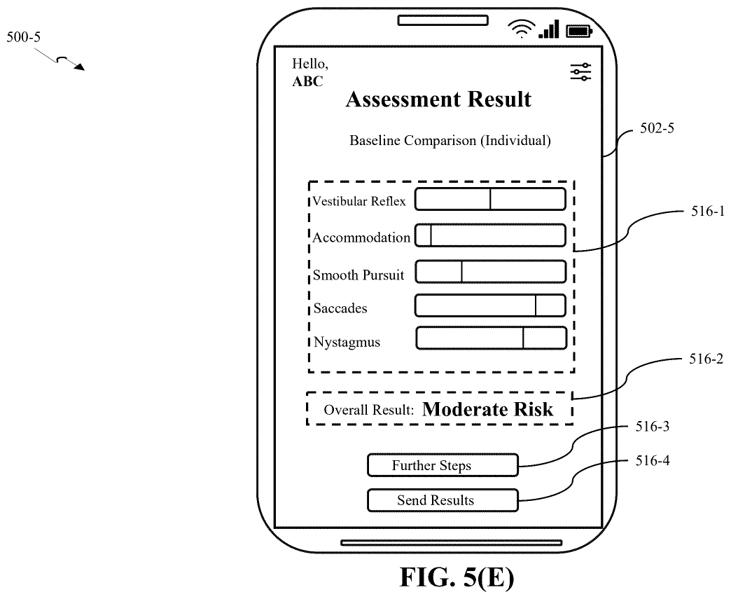

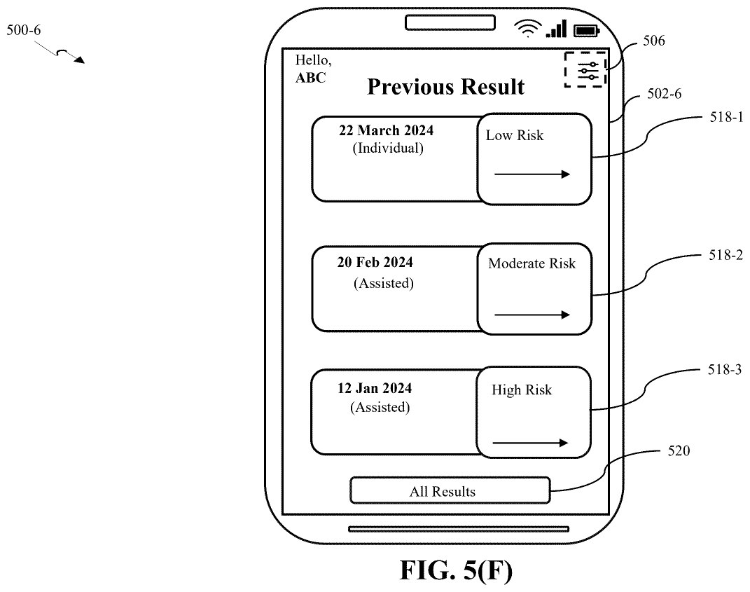

FIGS. 5(A)-5(F) illustrate Graphical User interfaces (GUIs) 500-1 through 500-6 presented through the user device corresponding to the neuro-ophthalmic risk assessment of the user, in accordance with an example aspect of the present disclosure.

Particularly, FIG. 5(A) illustrates example embodiments of the GUI 500-1 generated by the system 100 and presented by the user device 102 via an interface of the electronic application. The GUI 500-1 may include a screen 502-1 presented on the interface of the electronic application that facilitates the user 101 to select option(s) corresponding to the neuro-ophthalmic risk assessment. In the example embodiment, the screen 502-1 is shown to include elements 504, 506, 508-1, 508-2, and 508-3.

The element 504 may include the details of the user's account associated with the user device 102. Specifically, the element 504 may include a name of the user 101 operating the electronic application. In some aspects of the present disclosure, the element 504 may facilitate the user 101 to edit, manage, and/or view the user's account and setting(s) pertaining to permission(s) of the user's account. In some aspects of the present disclosure, the element 504 may be clickable such that a click on the element 504 may lead to another GUI (not shown) for editing, managing, and/or viewing the user's account and setting(s) pertaining to the permission(s) of the user account.

The element 506 may include selectable option(s) for the user 101. Preferably, the element 506 may include data sorting option(s) to apply data filter(s) on selection of data to be displayed on any of the elements 508-1 through 508-3. For example, the sorting option(s) in the element 506 may include sorting data based on date, size, rating, marker(s), and the like. Aspects of the present disclosure are intended to include or otherwise cover any type of filter(s) corresponding to providing filtering input(s) to the GUI 500-1 for the elements 508-1 through 508-3, without deviating from the scope of the present disclosure.

The element 508-1 may include selectable option(s) for the user 101. Preferably, the element 508-1 may include a Baseline Assessment option that enables the user 101 to initiate a baseline assessment test for neuro-ophthalmic risk assessment of the user. Specifically, the baseline assessment test option may facilitate the user 101 to provide baseline data (used to determine the baseline feature(s)) corresponding to an absence of the oculomotor impairments for the user 101. The baseline data may be used for deriving the baseline feature metrics that can be compared with the user metrics derived from the facial image(s) for the neuro-ophthalmic risk assessment of the user 101.

The element 508-2 may include selectable option(s) for the user 101. Preferably, the element 508-2 may include a Neuro-ophthalmic Assessment option that enables the user 101 to initiate a neuro-ophthalmic assessment test for neuro-ophthalmic risk assessment of the user 101. Specifically, the neuro-ophthalmic assessment test option may facilitate the user 101 to provide neuro-ophthalmic data (for reference) corresponding to an angle of impact and a value of force of impact on the user 101. The neuro-ophthalmic data may be used for deriving a neuro-ophthalmic feature metrics that can be compared with the user metrics derived from the facial image(s) for the neuro-ophthalmic risk assessment of the user 101.

The element 508-3 may include selectable option(s) for the user 101. Preferably, the element 508-3 may include a Previous test results option that enables historical risk assessment result(s) to be rendered to the user 101. Specifically, the element 508-3 may be clickable, such that a click on the element 504 may lead to another GUI (shown in GUI 500-6) for viewing and accessing the test results of the previous assessment test taken by the user 101.

FIG. 5(B) illustrates example embodiments of the GUI 500-2 generated by the system 100 and presented by the user device 102 via an interface of the electronic application. The GUI 500-2 may include a screen 502-2 presented on the interface of the electronic application that facilitates the user 101 to select option(s) corresponding to a type of neuro-ophthalmic risk assessment. In the example embodiment, the screen 502-2 is shown to include elements 510-1, and 510-2. The screen 502-2 may also include 504 and 506 (as shown in GUI 500-1).

The element 510-1 may include selectable option(s) for the user 101. Preferably, the element 510-1 may include an Individual Option that enables the user 101 to capture the facial image(s) for neuro-ophthalmic test(s). Specifically, the individual option may further render the demonstration element(s) for the neuro-ophthalmic test(s) selected by the user 101 to be rendered to the user 101 through user device 102.

The element 510-2 may include selectable option(s) for the user 101. Preferably, the element 510-2 may include an Assisted Option that enables the user 101 to be assisted by another user for capturing the facial image(s) of the user 101.

Specifically, the assisted option may further render demonstration element(s) (specific to the assisted mode) for the neuro-ophthalmic test(s) selected by the user 101 to be rendered to the other user through the user device 102.

FIG. 5(C) illustrates example embodiments of the GUI 500-3 generated by the system 100 and presented by the user device 102 via an interface of the electronic application. The GUI 500-3 may include a screen 502-3 presented on the interface of the electronic application that provides the demonstration element(s) to the user 101 to capture the facial image(s). In the example embodiment, the screen 502-3 is shown to include elements 512-1, 512-2, 512-3, and 512-4. The screen 502-3 may also include 504 and 506 (as shown in GUI 500-1).

The element 512-1 may include a display that runs a demonstration element (e.g., video clip) for a selected neuro-ophthalmic test. Preferably, the demonstration element guides the user 101 through a process of capturing the facial image(s) for the neuro-ophthalmic risk assessment test based on the options selected on GUI 500-1 and 500-2.

The element 512-2 may include selectable option(s). Preferably, the element 512-2 may facilitate the user 101 to control the demonstration element (i.e., play/pause the video clip).

The element 512-3 may include selectable option(s). Preferably, the element 512-3 may facilitate the user 101 to control the demonstration element (i.e., change the video to the next video on the element 512-1).

The element 512-4 may include selectable option(s). Preferably, the element 512-4 may facilitate the user 101 to control the demonstration element (i.e., change the current video playing to the previous video on the element 512-1).

FIG. 5(D) illustrates example embodiments of the GUI 500-4 generated by the system 100 and presented by the user device 102 via an interface of the electronic application. The GUI 500-4 may include a screen 502-4 presented on the interface of the electronic application that facilitates the user 101 to undergo neuro-ophthalmic test(s) (e.g., the accommodation test as presented in FIG. 5(D)). In the example embodiment, the screen 502-4 is shown to include element 514. The screen 502-4 may also include 504 and 506 (as shown in GUI 500-1).

The element 514 may include a letter to be presented on the screen 502-4 for the accommodation test. Specifically, the element 514 may enable the user to focus on the screen 502-4 to capture facial image(s) of the user for evaluating iris movement and head movement of the user 101.

FIG. 5(E) illustrates example embodiments of the GUI 500-5 generated by the system 100 and presented by the user device 102 via an interface of the electronic application. The GUI 500-5 may include a screen 502-5 presented on the interface of the electronic application that facilitates the user 101 to view the result of the neuro-ophthalmic risk assessment and further steps to take. In the example embodiment, the screen 502-5 is shown to include elements 516-1, 516-2, 516-3, and 516-4. The screen 502-5 may also include 504 and 506 (as shown in GUI 500-1).

The element 516-1 may render result(s) of the neuro-ophthalmic test(s) of the user 101. The result(s) may be rendered in any form (e.g., a bar graph, a comparative chart, a pie chart, a tabular representation, etc.) to the user 101. The element 516-2 may render the risk category of the user 101.

The element 516-3 may include selectable option(s). Preferably, the element 516-3 may include a Further Steps option for the user 101 to instruct the user 101 for measure(s) to be taken based on the risk category in the element 516-2. The element 516-3 may be clickable, such that a click on the element 516-3 may lead to another GUI (not shown).

The element 516-4 may include a selectable option. Preferably, the element 516-4 may include a Send Results option that facilitates the user 101 to share the neuro-ophthalmic risk assessment of the user 101 immediately with a physician for a timely treatment of the user 101. In some aspects of the present disclosure, the element 516-4 may be clickable, such that a click on the element 516-4 may lead to another GUI (not shown).

FIG. 5(F) illustrates an example embodiment of the GUI 500-6 generated by the system 100 and presented by the user device via an interface of the electronic application. The GUI 500-6 may include a screen 502-6 presented on the interface of the electronic application that facilitates the user 101 to view the results of the previous neuro-ophthalmic risk assessment taken by the user 101. In the example embodiment, the screen 502-6 is shown to include elements 518-1, 518-2, 518-3, and 520. The screen 502-5 may also include 504 and 506 (as shown in GUI 500-1).

The elements 518-1 through 518-3 may include selectable options. Preferably, the elements 518-1 through 518-3 may include an option to see the past result of a neuro-ophthalmic assessment of the user 101 matching the timeframe selected by the user 101. Specifically, the elements 518-1 through 518-3 may enable the user 101 to view the risk scores of neuro-ophthalmic test(s) of the user 101 and overall result(s) (e.g., risk assessment report(s)) of the neuro-ophthalmic risk assessment of the user 101. In some aspects of the present disclosure, the elements 518-1 through 518-3 may be clickable, such that a click on any of the elements 518-1 through 518-3 may lead to another GUI for displaying the results of the neuro-ophthalmic risk assessment test previously taken by the user 101.

The element 520 may include a selectable option. Preferably, the element 520 may include an All Results option to see result(s) of the historical neuro-ophthalmic test(s) previously taken by the user 101. In some aspects of the present disclosure, the element 506 may be used by the user 101 to select the timeline associated with the previous results.

As will be apparent to a person of ordinary skill in the art, the GUIs 500-1 through 500-6 presented by FIG. 5(A) through FIG. 5(F) are for illustration of some of the functions performed by the system 100, according to some example aspects of the present disclosure. It must be noted that the GUIs (as presented) does not signify a specific form of data presentation or providing information to or by the system 100 that may limit the scope of the present disclosure. Rather, the system 100 may generate any type of GUIs as may be suitable to render the various operations of the system 100 presented above.

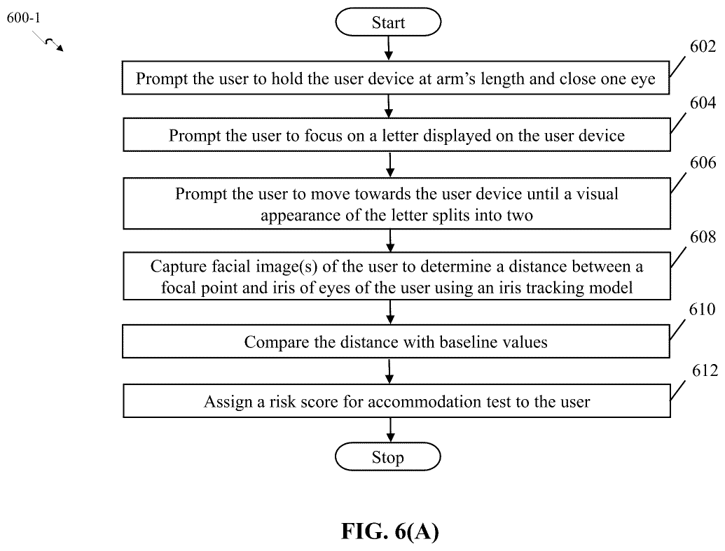

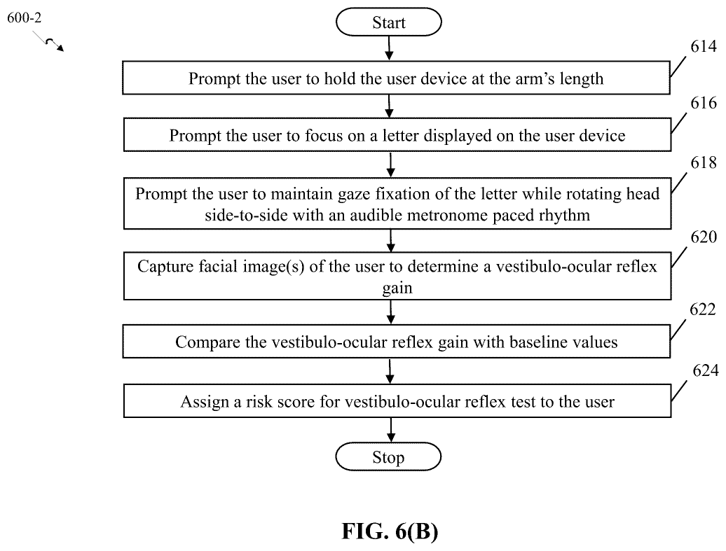

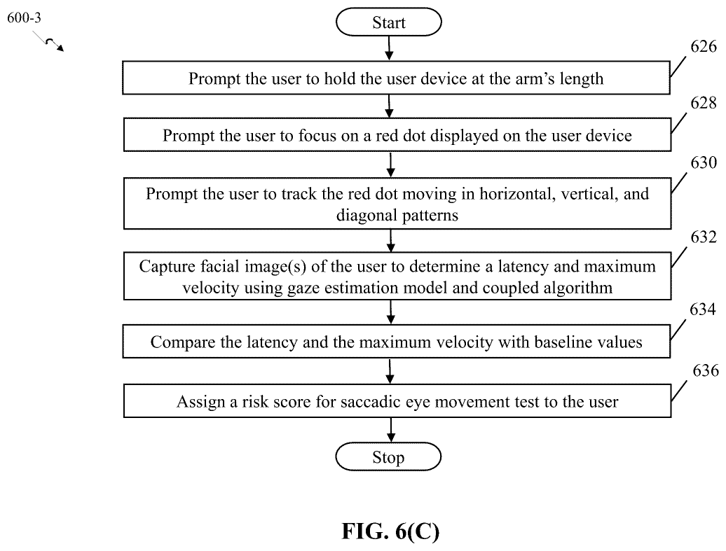

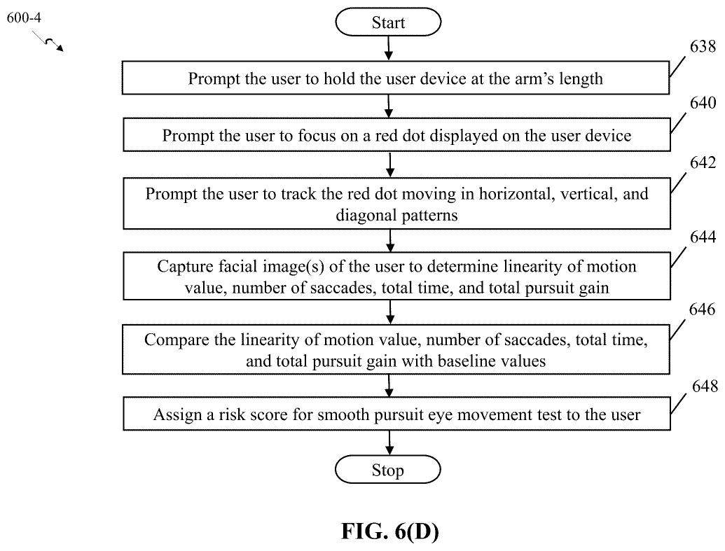

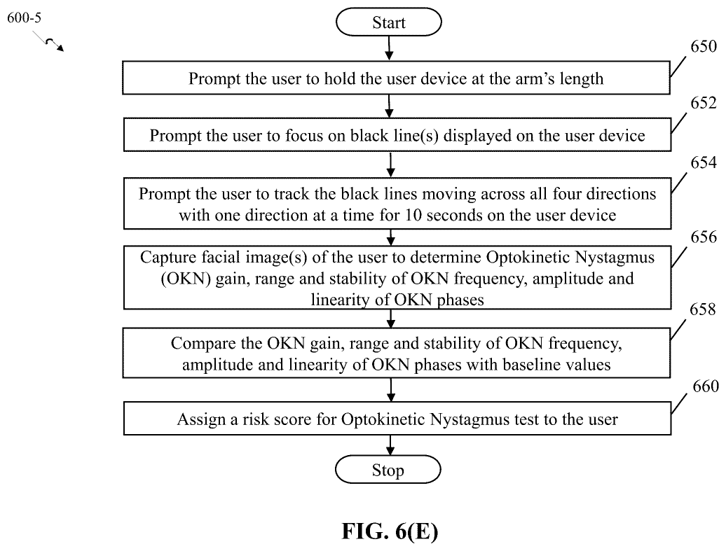

FIGS. 6(A)-6(E) illustrate process flow diagrams 600-1 through 600-5 depicting steps performed by the system 100 for various neuro-ophthalmic tests, in accordance with an example aspect of the present disclosure.

Particularly, FIG. 6(A) illustrates a process flow diagram 600-1 depicting steps performed by the system 100 for the accumulation test of the user 101, in accordance with an example aspect of the present disclosure. The process flow diagram comprises steps 602 through 612 rendered to the user 101 through the visual element(s).

At step 602, the system 100 may prompt the user 101 to hold the user device 102 at an arm's length and close one eye.

At step 604, the system 100 may prompt the user 101 to focus on a letter (e.g., ‘E’) displayed on the user device 102.

At step 606, the system 100 may prompt the user 101 to move towards the user device 102 until a visual appearance of the letter splits into two parts.

At step 608, the system 100 may facilitate the user 101 to capture the facial image(s) to determine a distance between a focal point and iris of the eyes of the user 101. In some aspects of the present disclosure, the system 100 may utilize an iris tracking model to determine the distance between the focal point and the iris of the eyes of the user 101. Specifically, the iris tracking model may be stored in the device memory 220 and may be executed through the device processor 210.

At step 610, the system 100 may compare the distance between the focal point and the iris of the eyes of the user 101 with baseline value(s) stored in the database 114.

At step 612, the system 100 may assign a risk score for the accommodation test to the user 101.

Particularly, FIG. 6(B) illustrates a process flow diagram 600-2 depicting steps performed by the system 100 for the VOR test of the user 101, in accordance with an example aspect of the present disclosure. The process flow diagram comprises steps 614 through 624 rendered to the user 101 through the visual element(s).

At step 614, the system 100 may prompt the user 101 to hold the user device 102 at the arm's length.

At step 616, the system 100 may prompt the user 101 to focus on the letter displayed on the user device 102.

At step 618, the system 100 may prompt the user 101 to maintain gaze fixation of the letter while rotating the head side-by-side with the audible metronome rhythm generated by the audio speaker(s) of the user device 102.

At step 620, the system 100 may facilitate the user 101 to capture the facial image(s). The system 100 may further determine a vestibulo-ocular reflex gain by analyzing the facial image(s).

At step 622, the system 100 may compare the vestibulo-ocular reflex gain with baseline value(s) of the vestibulo-ocular reflex gain stored in the database 114.

At step 624, the system 100 may assign a risk score for the VOR test to the user 101.

Particularly, FIG. 6(C) illustrates a process flow diagram 600-3 depicting steps performed by the system 100 for the saccadic eye movement test of the user 101, in accordance with an example aspect of the present disclosure. The process flow diagram comprises steps 626 through 636 rendered to the user 101 through the visual element(s).

At steps 626, the system 100 may prompt the user 101 to hold the user device 102 at the arm's length.

At step 628, the system 100 may prompt the user 101 to focus on a red dot displayed on the user device 102.

At step 630, the system 100 may prompt the user 101 to track the red dot moving in a horizontal pattern, a vertical pattern, and diagonal pattern(s) displayed on the user device 102.

At step 632, the system 100 may facilitate the user 101 to capture the facial image(s). The system 100 may further determine a latency value and a maximum velocity value based on an analysis of the facial image(s). In some aspects of the present disclosure, the system 100 may utilize a gaze estimation model and a coupled algorithm to determine the latency value and the maximum velocity value. Specifically, the gaze estimation model and the coupled algorithm may be stored in the device memory 220 and may be executed through the device processor 210.

At step 634, the system 100 may compare the latency value and the maximum velocity value with respective baseline value(s) stored in the database 114.

At step 636, the system 100 may assign a risk score for the saccadic eye movement test to the user 101.

Particularly, FIG. 6(D) illustrates a process flow diagram 600-4 depicting steps performed by the system 100 for the smooth eye pursuit test of the user 101, in accordance with an example aspect of the present disclosure. The process flow diagram comprises steps 638 through 648 rendered to the user 101 through the visual element(s).

At steps 638, the system 100 may prompt the user 101 to hold the user device 102 at the arm's length.

At step 640, the system 100 may prompt the user 101 to focus on the red dot displayed on the user device 102.

At step 642, the system 100 may prompt the user 101 to track the red dot moving in the horizontal pattern, the vertical pattern, and the diagonal pattern(s) displayed on the user device 102.

At step 644, the system 100 may facilitate the user 101 to capture the facial image(s). The system 100 may further determine a linearity of motion value, number of saccades, a total time value, and a total pursuit gain value based on an analysis of the facial image(s).

At step 646, the system 100 may compare the linearity of motion value, the number of saccades, the total time value, and the total pursuit gain value with corresponding baseline value(s) stored in the database 114.

At step 648, the system 100 may assign a risk score for the smooth pursuit eye movement test to the user 101.

Particularly, FIG. 6(E) illustrates a process flow diagram 600-5 depicting steps performed by the system 100 for the Optokinetic Nystagmus test of the user 101, in accordance with an example aspect of the present disclosure. The process flow diagram comprises steps 650 through 660 rendered to the user 101 through the visual element(s).

At steps 650, the system 100 may prompt the user 101 to hold the user device 102 at the arm's length.

At step 652, the system 100 may prompt the user 101 to focus on black line(s) displayed on the user device 102.

At step 654, the system 100 may prompt the user 101 to track the black line(s). the black line(s) may be displayed on the user device 102 moving across all four directions with one direction at a time for ten seconds.

At step 656, the system 100 may capture the facial image(s). The system 100 may further determine an Optokinetic Nystagmus (OKN) gain, a range value, a stability of OKN frequency value, an amplitude value, and a linearity of OKN phases value by analyzing the facial image(s).

At step 658, the system 100 may compare the OKN gain, the range value, the stability of OKN frequency value, the amplitude value, and the linearity of OKN phases value with corresponding baseline value(s) stored in the database 114.

At step 660, the system 100 may assign a risk score for the OKN test to the user 101.

As will be apparent to a person of ordinary skill in the art, the process flow diagrams 600-1 through 600-5 presented by FIG. 6(A) through FIG. 6(E) are for illustration of the neuro-ophthalmic test(s) performed by the system 100, according to some example aspects of the present disclosure. It must be noted that the process flow diagrams 600-1 through 600-5 (as presented) does not signify a specific form of data presentation or providing information to or by the system 100 that may limit the scope of the present disclosure.

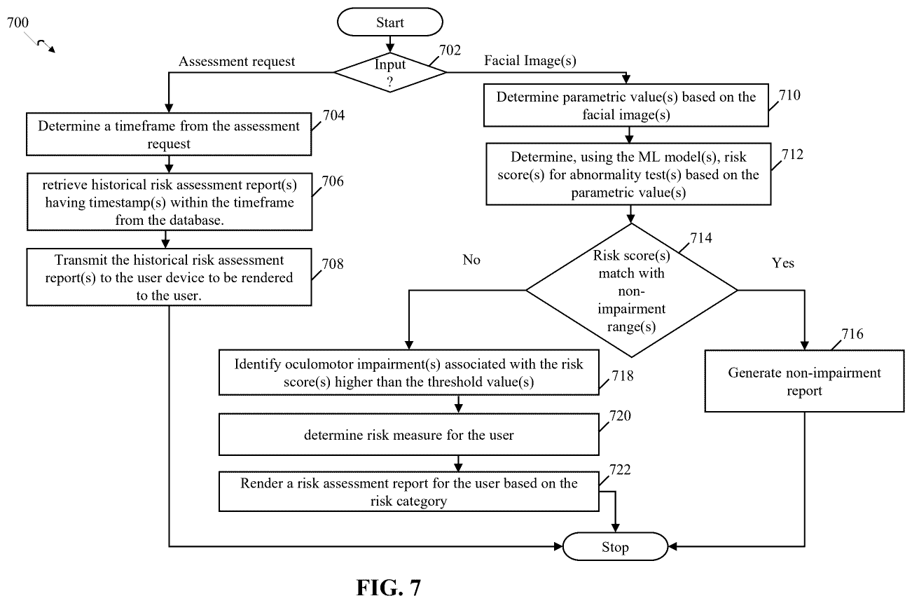

FIG. 7 is a flow chart that depicts a method 700 for neuro-ophthalmic risk assessment of the user 101, in accordance with an example aspect of the present disclosure. The method 700 may include various steps performed by the data processing server 104, depicted by way of blocks 702 through 722.

At block 702, the data processing server 104 may identify an input received from the user device 102. When the data processing server 104 identifies that the input received from the user device 102 is an assessment request for accessing the historical risk assessment report(s) of the user 101, the method 700 proceeds to block 704. Else, when the data processing server 104 identifies that the input received from the user device is in the form of the facial image(s) (i.e., corresponding to a present neuro-ophthalmic risk assessment of the user 101), the method 700 proceeds to block 710.

At block 704, the data processing server 104 may determine the timeframe associated with the assessment request to access the historical risk assessment report(s) of the user 101.

At block 706, the data processing server 104 may retrieve the historical risk assessment report(s) from the database having timestamp(s) matching the timeframe of the assessment request.

At block 708, the data processing server 104 may transmit the historical risk assessment report(s) to the user device 102 to be rendered to the user 101, and halt until a next input is received from the user device 102.

At block 710, the data processing server 104 may determine the parametric value(s) associated with the facial feature(s) of the user 101. In some aspects of the present disclosure, the facial feature(s) may be derived by analyzing the oculomotor reflexes of the user 101 through the facial image(s). In some aspects of the present disclosure, the facial feature(s) of the user 101 may be associated with tracking the movement of eyes of the user and estimating the gaze of the eyes of the user 101. In some aspects of the present disclosure, the data processing server 104 may identify pixel(s) from each of the facial image(s) comprising the facial feature(s) corresponding to the neuro-ophthalmic test(s). The data processing server 104 may further derive the user feature metrics using the facial feature(s) compare the user feature metrics with the baseline feature metrics to extract the parametric value(s).

At block 712, the data processing server 104 may identify the impairment level(s) for impairment(s) using the ML model(s) 108 based on the parametric value(s). The data processing server 104 may further determine the risk score(s) for the neuro-ophthalmic test(s) based on the parametric value(s) using the ML model(s) 108. In some aspects of the present disclosure, the neuro-ophthalmic test(s) comprises the accommodation test to measure the eye focus adjustment of the user 101, the VOR test to measure the stability of the eyes of the user 101, the saccadic eye movement test to evaluate the rapid gaze shift of the user 101, the smooth pursuit test to analyze continuous object tracking by the user 101, and the OKN test to analyze the succession of moving stimuli on the user 101. In some aspects of the present disclosure, the data processing server 104 may determine the risk score(s) by comparing the user feature metrics with the baseline feature metrics.

At block 714, the data processing server 104 may compare the risk score(s) with the non-impaired range. When the data processing server 104 determines that each of the risk score(s) are in the non-impaired range, the method 700 proceeds to block 716. Else, when the data processing server 104 determines that risk score(s) are not in the non-impaired range, the method 700 proceeds to block 718.

At block 716, the data processing server 104 may notify the user device with the non-impaired report of the user 101. The data processing server 104 may further determine the measure of risk for the user 101 based on the impairment level(s) of the user 101.

At block 718, the data processing server 104 may identify the oculomotor impairment(s) associated with the risk score(s). Moreover, the data processing server 104 may determine the measure of risk for the user 101 based on the impairment level(s) of the user 101.

At block 720, the data processing server 104 may assign the risk category to the user 101 based on the identified oculomotor impairment(s).

At block 722, the data processing server 104 may render the risk assessment report for the user 101 based on the risk category.

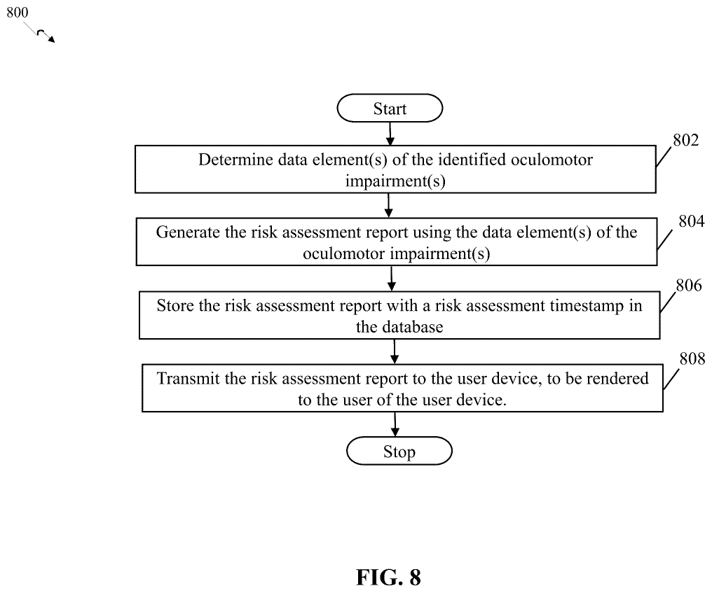

FIG. 8 is a flow chart that depicts a process 800 for rendering a risk assessment report of the user 101 (as depicted by the block 722), in accordance with an example embodiment of the present disclosure.

At block 802, the data processing server 104 may determine the data elements associated with information of the oculomotor impairment(s) based on the comparison of the user feature metrics derived from the facial image(s) with the baseline feature metrics.

At block 804, the data processing server 104 may generate the risk assessment report for the user 101 based on the data elements.

At block 806, the data processing server 104 may attach the risk assessment report with a timestamp and store the risk assessment report in the database 114.

At block 808, the data processing server 104 may transmit the risk assessment report to the user device 102.

Now, referring to the technical abilities and advantageous effect of the present disclosure, the system is independent of any specific configuration of a hardware for implementation, and the overall functionality is rather derived through the method. The system utilizes a user device (such as, but not limited to a smartphone, a tablet, a personal computer, or a laptop, etc.) to detect user's concussions based on quantifiable physical characteristics, thus eliminating the dependence on subjective evaluation, sports analysis, and outdoor activities monitoring. In another aspect of the present disclosure, the system facilitates the user to undergo the neuro-ophthalmic test(s) without a requirement of an external assistance.

Therefore, the system enables the neuro-ophthalmic test(s) to be self-administered which increases the accessibility of the system. Moreover, the system comprises data processing server that utilizes Machine Learning models to extract, analyze, and categorize unique metrics based on four unique tests to accurately diagnose a concussion. More particularly, the data processing server 104 is supported by two ML models. The first ML model is trained to extract feature metrics from the facial image(s) procured from each neuro-ophthalmic test. Specifically, the first ML model uses CNNs to derive the features. The second ML model is trained to compare the feature metrics with a baseline metric for neuro-ophthalmic assessments to determine the neuro-ophthalmic risk category. The second ML model. In some other aspects of the present disclosure, the system reduces latency for diagnosis of concussion(s).

The system further provides economic benefits to the user (that may be an injured patient) by early detection of the concussion(s) which facilitates the user to avail timely treatment for the detected concussion(s), thus avoiding any major expenditure in the future. The ease of use and availability of the system makes it accessible for the communities with lower income who may face concussions.

Those skilled in the art will appreciate that the methodology described herein in the present disclosure may be carried out in other specific ways than those set forth herein in the above disclosed embodiments without departing from essential characteristics and features of the technology. The above-described embodiments are therefore to be construed in all aspects as illustrative and not restrictive.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Any combination of the above features and functionalities may be used in accordance with one or more embodiments.

In the present disclosure, each of the embodiments has been described with reference to numerous specific details which may vary from embodiment to embodiment. The foregoing description of the specific embodiments disclosed herein may reveal the general nature of the embodiments herein that others may, by applying current knowledge, readily modify and/or adapt for various applications such specific embodiments without departing from the generic concept, and, therefore, such adaptations and modifications are intended to be comprehended within the meaning of the disclosed embodiments. It is to be understood that the phraseology or terminology employed herein is for the purpose of description and is not limited in scope.

What is claimed is:

1. A method for performing one or more neuro-ophthalmic risk assessments of a user, the method comprising:
receiving at least one facial image of the user from a user device;
determining, based on the at least one facial image through one or more neuro-ophthalmic tests, one or more parametric values associated with at least one facial feature of the user; and
determining a measure of risk for the user based on the one or more parametric values using one or more Machine Learning (ML) models, wherein the measure of risk indicates a level of neuro-ophthalmic risk associated with the user.

2. The method of claim 1, further comprises identifying, based on the one or more parametric values using the one or more ML models, one or more impairment levels for one or more impairments, wherein the one or more impairments are associated with the one or more neuro-ophthalmic tests.

3. The method of claim 1, wherein the at least one facial feature of the user is associated with tracking a movement of eyes of the user and estimating a gaze of the eyes of the user.

4. The method of claim 2, the method further comprising:
determining, using the one or more parametric values through the one or more ML models, one or more risk scores corresponding to the one or more neuro-ophthalmic tests;
determining whether at least one risk score from the one or more risk scores mismatches with a non-impaired range for a corresponding neuro-ophthalmic test from the one or more neuro-ophthalmic tests; and
identifying at least one oculomotor impairment from the one or more impairments based on the determination that the at least one risk score mismatches with the non-impaired range.

5. The method of claim 4, further comprises generating a non-impaired report based on the determination that each risk score from the one or more risk scores matches with the corresponding non-impaired range.

6. The method of claim 1, wherein the one or more neuro-ophthalmic tests comprises at least one of an accommodation test to measure an eye focus adjustment of the user, a Vestibulo-ocular Reflex (VOR) test to measure a stability of the eyes of the user, a saccadic eye movement test to evaluate a rapid gaze shift of the user, a smooth pursuit test to analyze continuous object tracking by the user, and an optokinetic nystagmus test to analyze response to a succession of moving stimuli on the user.

7. The method of claim 4, wherein the one or more ML models are trained for:
determining a user feature metrics corresponding to the one or more neuro-ophthalmic tests based on the one or more parametric values; and
comparing the user feature metrics with a baseline feature metrics to determine the one or more risk scores.

8. The method of claim 7, further comprising:
determining, based on the comparison of the user feature metrics with the baseline feature metrics, one or more data elements associated with information of the at least one oculomotor impairment;
generating a risk assessment report based on the one or more data elements;
storing the risk assessment report with a risk assessment timestamp in a database; and
transmitting the risk assessment report to the user device.

9. The method of claim 8, further comprising:
receiving an assessment request from the user device, wherein the assessment request corresponds to accessing at least one historical risk assessment report for a timeframe amongst one or more risk assessment reports stored in the database;
retrieving, from the database, the at least one historical risk assessment report having the risk assessment timestamp within the timeframe; and
transmitting the at least one historical risk assessment report to the user device.

10. The method of claim 1, further comprises rendering, through the user device, one or more demonstration elements for each neuro-ophthalmic test from the one or more neuro-ophthalmic tests.

11. A system to perform one or more neuro-ophthalmic risk assessments of a user, the system comprises:
a database; and
data processing circuitry coupled to the database, wherein the data processing circuitry is configured to:
receive at least one facial image of the user from a user device;
determine, based on the at least one facial image through one or more neuro-ophthalmic tests, one or more parametric values associated with at least one facial feature of the user; and
determine a measure of risk for the user based on the one or more parametric values using one or more Machine Learning (ML) models, wherein the measure of risk indicates a level of neuro-ophthalmic risk associated with the user.

12. The system of claim 11, wherein the data processing circuitry is further configured to identify, based on the one or more parametric values using the one or more ML models, one or more impairment levels for one or more impairments, wherein the one or more impairments are associated with the one or more neuro-ophthalmic tests.

13. The system of claim 11, wherein the at least one facial feature of the user is associated with tracking a movement of eyes of the user and estimating a gaze of the eyes of the user.

14. The system of claim 12, wherein the data processing circuitry is further configured to:
determine, using the one or more parametric values through the one or more ML models, one or more risk scores corresponding to the one or more neuro-ophthalmic tests;
determine whether at least one risk score from the one or more risk scores mismatches with a non-impaired range for a corresponding neuro-ophthalmic test from the one or more neuro-ophthalmic tests; and
identify at least one oculomotor impairment from the one or more impairments based on the determination that the at least one risk score mismatches with the non-impaired range.

15. The system of claim 11, wherein the one or more neuro-ophthalmic tests comprises at least one of an accommodation test to measure an eye focus adjustment of the user, a Vestibulo-ocular Reflex (VOR) test to measure a stability of the eyes of the user, a saccadic eye movement test to evaluate a rapid gaze shift of the user, a smooth pursuit test to analyze continuous object tracking by the user, and an optokinetic nystagmus test to analyze response to a succession of moving stimuli on the user.

16. The system of claim 14, wherein the data processing server, by way of one or more ML models, is configured to:
determine a user feature metrics corresponding to the one or more neuro-ophthalmic tests based on the one or more parametric values; and
compare the user feature metrics with a baseline feature metrics to determine the one or more risk scores.

17. The system of claim 16, wherein the data processing circuitry is further configured to:
determine, based on the comparison of the user feature metrics with the baseline feature metrics, one or more data elements associated with information of the at least one oculomotor impairment;
generate a risk assessment report based on the one or more data elements;
store the risk assessment report with a risk assessment timestamp in the database; and
transmit the risk assessment report to the user device.

18. The system of claim 17, wherein the data processing circuitry is further configured to:
receive an assessment request from the user device, wherein the assessment request corresponds to accessing at least one historical risk assessment report for a timeframe amongst one or more risk assessment reports stored in the database;
retrieve, from the database, the at least one historical risk assessment report having the risk assessment timestamp within the timeframe; and
transmit the at least one historical risk assessment report to the user device.

19. The system of claim 11, wherein the data processing circuitry is further configured to render, through the user device, one or more demonstration elements for each neuro-ophthalmic test from the one or more neuro-ophthalmic tests.

20. A computer program product for one or more neuro-ophthalmic risk assessments of a user, the computer program product comprising computer-executable instructions that are stored on a non-transitory computer-readable medium and that, when executed by a data processing circuitry performs operations comprising:
receiving at least one facial image of the user from a user device;
determining, based on the at least one facial image through one or more neuro-ophthalmic tests, one or more parametric values associated with at least one facial feature of the user; and
determining a measure of risk for the user based on the one or parametric values using one or more Machine Learning (ML) models, wherein the measure of risk indicates a level of neuro-ophthalmic risk associated with the user.
